(19) United States
(12) Patent Application Publication
(10) Pub. No.: US 2008/0187946 A1
(43) Pub. Date: Aug. 7, 2008

(54) APPARATUS FOR AND METHOD OF DETECTING MICROORGANISM OR MICRO-PARTICLE IN REAL TIME BY USING ELECTRICAL CHARGING METHOD

(75) Inventors: Jin Sang HWANG, Suwon-si (KR); Jung Joo HWANG, Suwon-si (KR); Mi Jeong SONG, Suwon-si (KR)

Correspondence Address: CANTOR COLBURN, LLP, 20 Church Street, 22nd Floor, Hartford, CT, 06103, US

(73) Assignee: SAMSUNG ELECTRONICS CO., LTD, Suwon-si (KR)

(21) Appl. No.: 11/776,963

(22) Filed: Jul. 12, 2007

(30) Foreign Application Priority Data
Oct. 16, 2006 (KR) 10-2006-0100300

(51) Int. Cl. C12Q 1/20; C12M 1/34; G06F 19/00

(52) U.S. Cl. 435/29; 435/288.7; 435/286.7; 702/19

(57) ABSTRACT

An apparatus for detecting a microorganism or micro-particle in real time includes an electrical charging module which electrically charges a microorganism or micro-particle in an atmosphere, a collection module which collects the electrically charged microorganism or micro-particle by using a potential difference, a preprocessing module which applies a fluorescent material to the collected microorganism or micro-particle and a sensing module which irradiates a light, senses a microorganism or micro-particle which reacts to the light and detects a concentration of the microorganism or micro-particle in the atmosphere.

### CROSS-REFERENCE TO RELATED APPLICATION

This application claims priority to Korean Patent Application No. 10-2006-0100300, filed on Oct. 16, 2006, and all the benefits accruing therefrom under 35 U.S.C. §119, the contents of which in its entirety are herein incorporated by reference.

### BACKGROUND OF THE INVENTION

1. Field of the Invention

The present invention relates to an apparatus for and a method of detecting a microorganism or micro-particle, and more particularly, to an apparatus for and a method of detecting a microorganism or micro-particle in real time using an electrical charging method.

2. Description of Related Art

Numerous microorganisms such as bacteria, viruses and molds float in air, and humans are directly or indirectly affected by these microorganisms. A typical apparatus for detecting a microorganism or micro-particle of a particular size in the air in real time includes an air-conditioner, an air cleaner, environment biosensors and the like.

The above-described apparatus which uses a conventional microorganism measurement method requires culturing for at least a few hours to a few days in a culture medium. The above-described apparatus may also be used with a professional analysis method which is performed in a solution environment, e.g., polymerase chain reaction (“PCR”), an antigen-antibody reaction.

Specifically, research on or monitoring microorganisms in the air has been generally conducted in laboratories, hospitals or pharmaceutical factories where attention to microbe infection is required. In order to measure microorganisms in the air, collecting samples for research and for measuring an amount of or types of microorganisms from the collected samples is required. However, the conventional microorganism measurement method requires a significant amount of time and effort, and requires additional time to culture the collected samples.

The conventional microorganism measurement method may be classified into two methods: a method of directly analyzing microorganisms or living bodies which show biological characteristics; and a method of analyzing metabolites of the microorganisms. The method of directly analyzing microorganisms or living bodies is typically used, and includes a culture method, a cell count method, a PCR method, a flow cell calculation method, an adenosine-3-triphosphate (“ATP”) detection method and the like.

In the culture method, samples are directly cultured in a culture medium, the number of formed colonies is counted, and thus a result of whether microorganisms exist is ascertained. However, more than 24 hours are typically expended in culturing the samples, and a period of over seven days are expended in detecting fungi. Therefore, microorganisms may not be quickly detected. In addition, certain microorganisms within the collected samples may not be detected, since a few types of bacteria may not be cultured by typical methods. Moreover, colony formation may be affected by several factors such as types of microorganisms, formation of the culture medium, time allowed for a culture, temperature, humidity and the like. Thus, the conventional microorganism measurement methods described above require a culture operation for acquiring several samples from the collected samples of the microorganisms. In addition, microorganisms which may be measured are limited to only those types of microorganisms which may be cultured.

A microorganism measurement method based on an electrical method has been previously developed. However, research on an advanced collection operation, which applies an electrical method, has not been developed. Furthermore, the current electrical method typically requires fluid samples, and thus may not be suitable for the measurement of microorganisms.

### BRIEF SUMMARY OF THE INVENTION

An exemplary embodiment of the present invention provides an apparatus for and a method of detecting a microorganism or micro-particle in real time which may perform a collecting as well as a sensing operation of the microorganism or micro-particle in real time.

An exemplary embodiment of the present invention also provides an apparatus for and a method of detecting a microorganism or micro-particle in real time which may sense the microorganism or micro-particle by using an electrical charging method without directly culturing samples in a culture medium.

According to an exemplary embodiment of the present invention, there is provided an apparatus for detecting a microorganism or micro-particle in real time, the apparatus includes an electrical charging module which electrically charges a microorganism or micro-particle in an atmosphere, a collection module which collects the electrically charged microorganism or micro-particle by using a potential difference, a preprocessing module which applies a fluorescent material to the collected microorganism or micro-particle and a sensing module which irradiates a light, senses a microorganism or micro-particle which reacts to the light and detects a concentration in the atmosphere.

According to another exemplary embodiment of the present invention, there is provided a method of detecting a microorganism or micro-particle in real time, the method includes electrically charging a microorganism or micro-particle in an atmosphere, collecting the electrically charged microorganism or micro-particle by using a potential difference, preprocessing the collected microorganism or micro-particle by applying a fluorescent material to the collected microorganism or micro-particle and irradiating a light, sensing a microorganism or micro-particle which reacts to the light and detecting a concentration of the microorganism or micro-particle in the atmosphere Additional and/or other aspects, features and advantages of the present invention will be set forth in part in the description which follows and, in part, will be obvious from the description, or may be learned by practice of the invention.

### BRIEF DESCRIPTION OF THE DRAWINGS

The above and/or other aspects, features and advantages of the present invention will become apparent and more readily appreciated from the following detailed description, taken in conjunction with the accompanying drawings, in which:

FIG. 1 is a block diagram illustrating a configuration of an exemplary embodiment of an apparatus for detecting a microorganism or micro-particle in real time by using an electrical charging method according to the present invention;

FIG. 2 is a front perspective diagram view illustrating an exemplary embodiment of an apparatus for detecting a microorganism or micro-particle in real time by using an electrical charging method according to the present invention;

FIG. 3 is a front diagram view illustrating a second exemplary embodiment of a cleaning module according to the present invention; and

FIG. 4 is a flowchart illustrating an exemplary embodiment of a method of detecting a microorganism or micro-particle in real time by using an electrical charging method according to the present invention.

### DETAILED DESCRIPTION OF THE INVENTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to cross section illustrations that are schematic illustrations of idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present invention.

Reference will now be made in detail to exemplary embodiments of the present invention, examples of which are illustrated in the accompanying drawings, wherein like reference numerals refer to the like elements throughout. The exemplary embodiments are described below in order to explain the present invention by referring to the figures.

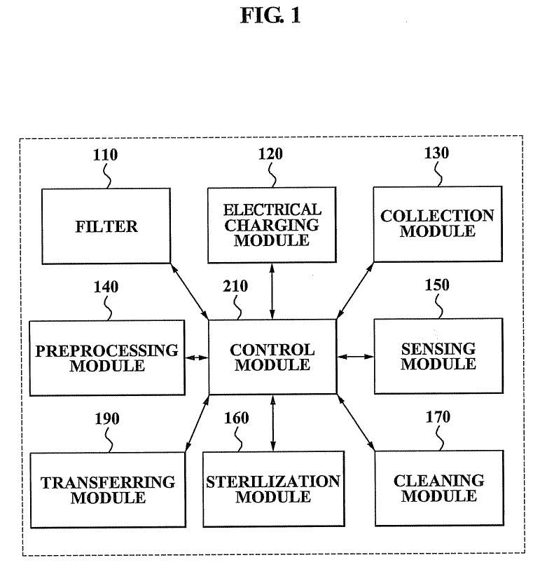

FIG. 1 is a block diagram illustrating a configuration of an exemplary embodiment of an apparatus for detecting a microorganism or micro-particle in real time by using an electrical charging method according to the present invention.

Referring to FIG. 1, the exemplary apparatus for detecting a microorganism or micro-particle in real time by using an electrical charging method according to the present invention includes a control module 210, an electrical charging module 120, a collection module 130, a preprocessing module 140 and a sensing module 150.

The electrical charging module 120 electrically charges the microorganism or micro-particle which is introduced into the apparatus for detecting a microorganism or micro-particle in real time in an atmosphere. In exemplary embodiments, the electrical charging module 120 may electrically charge the microorganism or micro-particle by attaching a positive ion or a negative ion, which is generated by a positive corona discharge or a negative corona discharge, to the microorganism or micro-particle. In further exemplary embodiments, the electrical charging module 120 may correspond to any type of module or apparatus which may be appreciated by those skilled in the art.

The collection module 130 collects the electrically charged microorganism or micro-particle by using a potential difference. In this instance, the collection module 130 may generate a potential difference including a predetermined value across the microorganism or micro-particle, and collects the microorganism or micro-particle which is attracted towards a collection medium 133. In this instance, the collection module 130 may generate a potential difference of a predetermined value between a collection medium 133 and the microorganism or micro-particle, and collect the microorganism or micro-particle attracted towards the collection medium 133. The collection module 130 includes a circulation belt 131 and a collection roller 132. The circulation belt 131 includes the collection medium 133 on which the microorganism or micro-particle adheres to. The collection roller 132 drives the circulation belt 131. The collection roller 132 is in electrical contact with the collection medium 133 via the circulation belt 131. Also, a predetermined potential difference is applied to the collection roller 132 in order to enable the microorganism or micro-particle to be transferred to a transferring module 190.

The preprocessing module 140 applies a fluorescent material to the microorganism or micro-particle which is collected in the collection module 130. In an exemplary embodiment, a fluorescent antibiotic is used as the fluorescent material. The fluorescent antibiotic is a new material wherein the fluorescent material adheres to antibiotics. In further exemplary embodiments, Fluorescein I tagged-Vancomycin, Fluorescein II tagged-Vancomycin, BodipyF1 tagged-Vancomycin, Fluorescein I tagged-Ramoplanin, Fluorescein II tagged-Ramoplanin and BodipyF1 tagged-Ramoplanin may be used as the fluorescent material.

The sensing module 150 irradiates a light, senses a microorganism or micro-particle which reacts to the irradiated light and thereby detects a concentration of the microorganism or micro-particle in the atmosphere. In exemplary embodiments, the microorganism or micro-particle which is collected in the collection module 130 may be counted by the sensing module 150.

According to an exemplary embodiment of the present invention, the apparatus for detecting a microorganism or micro-particle in real time by using an electrical charging method may sense the microorganism or micro-particle without directly culturing samples in a culture medium.

In exemplary embodiments, the apparatus for detecting a microorganism or micro-particle in real time by using an electrical charging method may further include a filter 110, a transferring module 190, a sterilization module 160, a cleaning module 170 and a post processing module, which is not illustrated.

The filter 110 filters a microorganism or micro-particle which is larger than a predetermined size from the atmosphere, and guides the filtered microorganism or micro-particle which is smaller than or equal to the predetermined size to the electrical charging module 120. In exemplary embodiments, the transferring module 190 transfers the collected microorganism or micro-particle to a transfer belt (not shown). In an exemplary embodiment, the transferring module 190 may include a circulation belt 191 and a transfer roller 192. The microorganism or micro-particle is transferred and adhered to the circulation belt 191. Also, the transfer roller 192 drives the circulation belt 191, and applies a predetermined potential difference to the circulation belt 191 in order to enable the microorganism or micro-particle to be transferred to the transferring module 190.

In exemplary embodiments, the sterilization module 160 may further include a first sterilization module 180 and a second sterilization module 280. The first sterilization module 180 sterilizes the microorganism or micro-particle which is adhered to the collection module 130 and which is not transferred to the transferring module 190. The second sterilization module 280 sterilizes the microorganism or micro-particle transferred to the transferring module 190. In exemplary embodiments, the first sterilization module 180 and the second sterilization module 280 may include one of an ultraviolet lamp and a sterilization filter. However, the present invention is not limited thereto.

Also, in exemplary embodiments, the cleaning module 170 may farther include a first cleaning module (not shown) and a second cleaning module 270. The first cleaning module separates the microorganism or micro-particle from the collection module 130 which is not transferred from the collection module 130 to the transferring module 190. The second cleaning module 270 separates the microorganism or micro-particle from the transferring module 190. Also, in further exemplary embodiments, the first cleaning module may include a conductive substrate and a cleaning blade. The conductive substrate applies a predetermined charging voltage. The cleaning blade removes the microorganism or micro-particle which remains in the collection module 130 by applying the charging voltage via the conductive substrate. In exemplary embodiments, the second cleaning module 270 may include a conductive substrate 171 and a cleaning blade 172. The conductive substrate 171 applies a predetermined charging voltage. The cleaning blade 172 removes the microorganism or micro-particle which remains in the transferring module 190 by applying the charging voltage via the conductive substrate 171.

Also, the post processing module controls a driving speed of the circulation belts 131 and 191 according to a concentration of the microorganism or micro-particle.

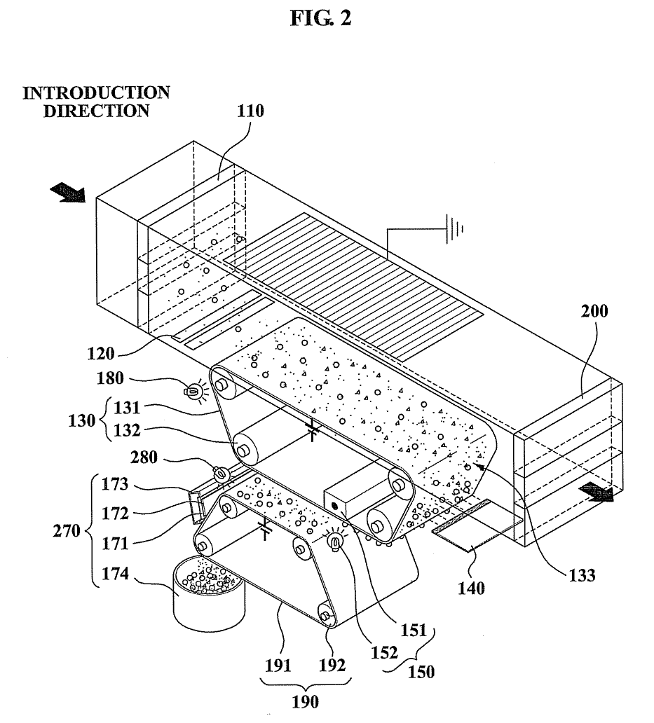

FIG. 2 is a front perspective diagram view illustrating an exemplary embodiment of an apparatus for detecting a microorganism or micro-particle in real time by using an electrical charging method according to the present invention.

Referring to FIG. 2, the microorganism or micro-particle including a predetermined size in an atmosphere is introduced into the apparatus for detecting a microorganism or micro-particle in real time by using an electrical charging method. A filter 110 filters out a microorganism or micro-particle which is larger than a predetermined size from the atmosphere. The pore size of the filter 110 which filters the microorganism or micro-particle is between about 0.5 μm and about 10 μm. Therefore, a filtered microorganism or micro-particle, which includes a size between about 0.5 μm and about 10 μm, passes through the filter 110 and an electrical charging module 120, and is thereby electrically charged to a predetermined polarity. The electrical charging module 120 corresponds to an apparatus for electrically charging a microorganism or micro-particle by attaching a positive ion or a negative ion, which is generated by a positive corona discharge or a negative corona discharge, to the microorganism or micro-particle. However, the electrical charging module 120 of the present invention is not limited to a corona discharge method.

The electrically charged microorganism or micro-particle is collected by a collection module 130 by using a potential difference. Specifically, a potential difference which includes a predetermined value is generated across the electrically charged microorganism or micro-particle. Thus, the microorganism or micro-particle is thereby attracted towards and collected by a collection medium 133. The collection module 130 according to an exemplary embodiment of the present invention includes a circulation belt 131 and a collection roller 132. The circulation belt 131 includes the collection medium 133 on which the microorganism or micro-particle adheres to, and the collection roller 132 which drives the circulation belt 131. In addition, the collection roller 132 is in electrical contact with the collection medium 133 via the circulation belt 131. A predetermined potential difference is applied to the collection roller 132 in order to enable the microorganism or micro-particle to be transferred to a transferring module 190 from the collection module 130. In FIG. 2, a voltage formation module (not illustrated) for forming the predetermined potential difference is embodied in the circulation belt 131. However, in exemplary embodiments, the predetermined potential difference may be formed via the collection roller 132. The microorganism or micro-particle is collected on the circulation belt 131 including the collection medium 133. The microorganism or micro-particle which is collected onto the circulation belt 131 circulates and passes a subsequent module.

The microorganism or micro-particle, which is collected in the collection module 130, passes a preprocessing module 140 and the preprocessing module 140 applies a fluorescent material to the collected microorganism or micro-particle. In an exemplary embodiment, a fluorescent antibiotic is used as the fluorescent material. The fluorescent antibiotic is a new material wherein the fluorescent material adheres to an antibiotic. In further exemplary embodiments, Fluorescein I tagged-Vancomycin, Fluorescein II tagged-Vancomycin, BodipyF1 tagged-Vancomycin, Fluorescein I tagged-Ramoplanin, Fluorescein II tagged-Ramoplanin and BodipyF1 tagged-Ramoplanin may be used as the fluorescent material. The fluorescent material is a material which strongly adheres to a particular microorganism or micro-particle and does not strongly adhere to other materials. In exemplary embodiments, an adsorptive ability of the microorganism or micro-particle may be detected by a predetermined fluorescent color as a user desires.

The sensing module 150 irradiates a light, senses a microorganism or micro-particle which reacts to the irradiated light and thereby detects a concentration of the microorganism or micro-particle in the atmosphere. In exemplary embodiments, the sensing module 150 may detect and count a number of the microorganisms or micro-particles which is collected by the collection module 130 by sensing a light from the microorganism or micro-particle which reacts with the irradiated light. In further exemplary embodiments, the sensing module 150 may include a light source 152 and a detector 151. The light source 152 irradiates the light and the detector 151 detects a concentration of the microorganism or micro-particle and counts a number of the microorganisms or micro-particles collected.

The microorganism or micro-particle passes to the transferring module 190. The transferring module 190 transfers the collected microorganism or micro-particle to a transfer belt (not shown).

The microorganism or micro-particle is collected by the collection module 130 by creating a potential difference, and the microorganism or micro-particle thereby adheres to the collection medium 133 on the circulation belt 131. In an exemplary embodiment, the potential difference is larger in the transferring module 190 than the potential energy in the collection module 130. In an exemplary embodiment, the transferring module 190 may include the transfer belt 191 and a transfer roller 192. The microorganism or micro-particle is transferred and adhered to the circulation belt 191. The transfer roller 192 drives the circulation belt 191, and applies the predetermined potential difference in order to enable the microorganism or micro-particle to be transferred to the transferring module 190. In FIG. 2, although the voltage formation module for creating the potential difference, which is larger than the potential difference in the collection module 130, is located on the transfer belt 191, the voltage formation module may be embodied on the transfer roller 192 in exemplary embodiments.

The transferred microorganism or micro-particle passes to a sterilization module 160. In exemplary embodiments, the sterilization module 160 may include a first sterilization module 180 and a second sterilization module 280. The first sterilization module 180 sterilizes the microorganism or micro-particle which adheres to the collection module 130 and which is not transferred to the transferring module 190. The second sterilization module 280 sterilizes the microorganism or micro-particle transferred to the transferring module 190. Specifically, the first sterilization module 180 removes the microorganism or micro-particle which remains on a collection belt 131. The second sterilization module 280 removes the microorganism or micro-particle which is used in order to complete the detecting and counting of the microorganism or micro-particle via the sensing module 150, and which is transferred to the transferring module 190. In exemplary embodiments, the first sterilization module 180 and the second sterilization module 280 may include one of an ultraviolet lamp and a sterilization filter. However, the present invention is not limited thereto.

Also, the microorganism or micro-particle, which is not removed by the first sterilization module 180 and the second sterilization module 280, is removed via a cleaning module 170. In exemplary embodiments, the cleaning module 170 may include a first cleaning module, which is not illustrated, or a second cleaning module 270. The first cleaning module separates the microorganism or micro-particle, which is not transferred from the collection module 130 to the transferring module 190, from the collection module 130. In an exemplary embodiment, the first cleaning module may include a conductive substrate and a cleaning blade. The conductive substrate applies a predetermined charging voltage. The cleaning blade removes the microorganism or micro-particle which remains in the collection module 130 by applying the charging voltage via the conductive substrate. Also, the second cleaning module 270 separates the microorganism or micro-particle from the transferring module 190. In an exemplary embodiment, the second cleaning module 270 may include a conductive substrate 171 and a cleaning blade 172. The conductive substrate 171 applies a predetermined charging voltage. The cleaning blade 172 removes the microorganism or micro-particle which remains on the transferring module 190 by applying the charging voltage via the conductive substrate 171. In exemplary embodiments, the microorganism or micro-particle which is separated from the transferring module 190 by the second cleaning module 270 may be collected by a separate collection container 174 included in the second cleaning module 270.

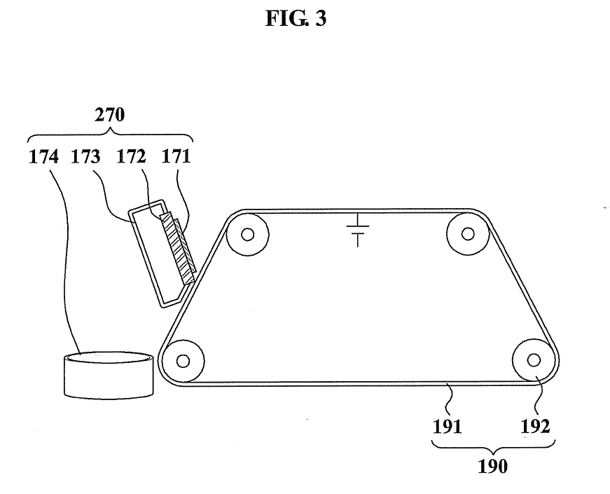

FIG. 3 is a front diagram view illustrating a second exemplary embodiment of a cleaning module according to the present invention. The second cleaning module 270 includes a conductive substrate 171, a cleaning blade 172, a collection container 173 and a separate collection container 174. The conductive substrate 171 is spaced apart from a transfer belt 191 by a predetermined distance, and electrically charges the transfer belt 191 to a particular polarity by applying a charging voltage. The cleaning blade 172 is integrally formed with the conductive substrate 171 and installed to be in contact with the transfer belt 191. Also, the cleaning blade 172 is supplied with the charging voltage via the conductive substrate 171, to electrically charge the transfer belt 191 via a particular electrode, and removes the microorganism or micro-particle which remains on the transfer belt 191. The collection container 173 collects the microorganism or micro-particle which is removed from the transfer belt 191. In the current exemplary embodiment, a separate collection container 174 may collect the microorganism or micro-particle which is removed from the transfer belt 191.

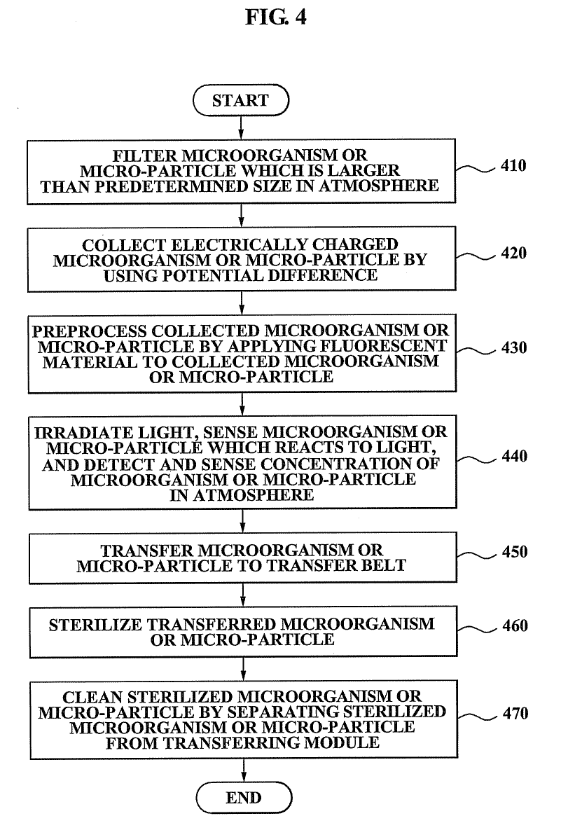

FIG. 4 is a flowchart illustrating an exemplary embodiment of a method of detecting a microorganism or micro-particle in real time by using an electrical charging method according to the present invention.

Referring to FIG. 4, in operation 410, a microorganism or micro-particle which is larger than a predetermined size in an atmosphere is filtered. In this instance, a filter 110 which filters the microorganism or micro-particle includes a pore size between about 0.5 μm and about 10 μm.

In operation 420, the microorganism or micro-particle in the atmosphere is electrically charged and the electrically charged microorganism or micro-particle is collected by using a potential difference. Specifically, a potential difference which includes a predetermined value is generated across the electrically charged microorganism or micro-particle. Thus, the microorganism or micro-particle is thereby attracted towards and collected at a collection medium 133.

In operation 430, the collected microorganism or micro-particle is preprocessed by applying a fluorescent material to the collected microorganism or micro-particle. In operation 440, a light is irradiated onto the microorganism or micro-particle, and a microorganism or micro-particle which reacts to the irradiated light is sensed and thereby a concentration of the microorganism or micro-particle in the atmosphere is detected and sensed. The fluorescent material is a material which strongly adheres to a particular microorganism or micro-particle and does not strongly adhere to other materials. In an exemplary embodiment, an adsorptive ability for the microorganism or micro-particle may be detected by a predetermined fluorescent color as a user desires. Also, through the sensing operation described above, the collected microorganism or micro-particle may be detected and counted by sensing a light from the microorganism or micro-particle which reacts with the irradiated light.

In operation 450, the microorganism or micro-particle is transferred to a transfer belt (not shown). Specifically, a potential difference, which includes a larger value than the potential difference created when collecting the microorganism or micro-particle, is created, and the microorganism or micro-particle which is adhered to the collection medium 133 on a circulation belt 131, is thereby transferred to the transfer belt.

In operation 460, the transferred microorganism or micro-particle is sterilized. In operation 460, a first sterilizing operation and a second sterilizing operation may be performed in exemplary embodiments. In the first sterilizing operation, the microorganism or micro-particle which is adhered to a collection module 130 is sterilized. In the second sterilizing operation, the microorganism or micro-particle which is transferred to the transferring module 190 is sterilized. In the first sterilizing operation and the second sterilizing operation, one of an ultraviolet lamp and a sterilization filter is used for sterilizing. However, the present invention is not limited thereto.

In operation 470, the sterilized microorganism or micro-particle is cleaned by separating the sterilized microorganism or micro-particle from a transferring module 190. In operation 470, a first cleaning operation and second cleaning operation may be performed in exemplary embodiments. In the first cleaning operation, the microorganism or micro-particle, which is not transferred from the collection module 130 to the transferring module 190, is separated from the collection module 130. In the second cleaning operation, the microorganism or micro-particle is separated from the transferring module 190.

The method of detecting a microorganism or micro-particle in real time according to the above-described exemplary embodiment of the present invention may be recorded in computer-readable media including program instructions in order to implement various operations embodied by a computer. In exemplary embodiments, the media may also include, alone or in combination with the program instructions, data files, data structures and the like. In further exemplary embodiments, the media and program instructions may be those specially designed and constructed for the purposes of the present invention, or they may be of the type well-known and available to those having ordinary skill in the computer software arts. Exemplary embodiments of computer-readable media include magnetic media such as hard disks, floppy disks and magnetic tape; optical media such as CD ROM disks and DVD; magneto-optical media such as optical disks; and hardware devices which are specially configured to store and perform program instructions, such as read-only memory (“ROM”), random access memory (“RAM”), flash memory and the like. In exemplary embodiments, the media may also be a transmission medium such as optical or metallic lines, wave guides and the like, including a carrier wave which transmit signals specifying the program instructions, data structures and the like. Exemplary embodiments of program instructions include both machine code, such as produced by a compiler, and files containing higher level code which may be executed by the computer using an interpreter. In exemplary embodiments, the described hardware devices may be configured to act as one or more software modules in order to perform the operations of the above-described exemplary embodiments of the present invention.

An apparatus for and a method of detecting a microorganism or micro-particle in real time according to the above-described exemplary embodiments of the present invention may perform collecting as well as sensing operations of the microorganism or micro-particle in real time.

An apparatus for and a method of detecting a microorganism or micro-particle in real time according to the above-described exemplary embodiments of the present invention may sense the microorganism or micro-particle by using an electrical charging method without directly culturing samples in a culture medium.

Although a few embodiments of the present invention have been shown and described, the present invention is not limited to the described exemplary embodiments. Instead, it would be appreciated by those of ordinary skill in the art that changes may be made to these exemplary embodiments without departing from the principles and spirit of the present invention, the scope of which is defined by the claims and their equivalents.

What is claimed is:

1. An apparatus for detecting a microorganism or micro-particle in real time, the apparatus comprising:
an electrical charging module which electrically charges a microorganism or micro-particle in an atmosphere;
a collection module which collects the electrically charged microorganism or micro-particle by using a potential difference;
a preprocessing module which applies a fluorescent material to the collected microorganism or micro-particle; and
a sensing module which irradiates a light, senses a microorganism or micro-particle which reacts to the light and detects a concentration of the microorganism or micro-particle in the atmosphere.

2. The apparatus of claim 1, further comprising:
a filter which filters a microorganism or micro-particle which is larger than a predetermined size in the atmosphere, and which guides the filtered microorganism or micro-particle to the electrical charging module.

3. The apparatus of claim 1, further comprising:
a transferring module which transfers the collected microorganism or micro-particle to a transfer belt.

4. The apparatus of claim 1, further comprising:
a circulation belt including a collection medium which adheres to the microorganism or micro-particle; and
a collection roller where a potential difference is applied to enable the microorganism or micro-particle to be transferred to the transferring module.

5. The apparatus of claim 4, further comprising:
a post processing module which controls a driving speed of the circulation belt according to the concentration of the microorganism or micro-particle.

6. The apparatus of claim 1, further comprising:
a first sterilization module which sterilizes the microorganism or micro-particle which is adhered to the collection module; and
a first cleaning module which separates the microorganism or micro-particle which is not transferred from the collection module to the transferring module, from the collection module.

7. The apparatus of claim 6, wherein the first cleaning module comprises:
a conductive substrate which applies a predetermined charging voltage; and
a cleaning blade which removes the microorganism or micro-particle which remains in the collection module by applying the charging voltage via the conductive substrate.

8. The apparatus of claim 3, further comprising:
a second sterilization module which sterilizes the transferred microorganism or micro-particle; and
a second cleaning module which separates the microorganism or micro-particle from the transferring module.

9. The apparatus of claim 8, wherein the second cleaning module comprises:
a conductive substrate which applies a predetermined charging voltage; and
a cleaning blade which removes the microorganism or micro-particle which remains in the transferring module by applying the charging voltage via the conductive substrate.

10. The apparatus of claim 2, wherein the filter which filters the microorganism or micro-particle is between about 0.5 μm and about 10 μm.

11. The apparatus of claim 1, wherein the sensing module counts a number of microorganisms or micro-particles to which the fluorescent material is applied, and detects a concentration of the microorganism or micro-particle in the atmosphere.

12. The apparatus of claim 3, wherein the transferring module further comprises:
a circulation belt to which the microorganism or micro-particle is transferred and adhered; and
a transfer roller which drives the circulation belt, and applies a potential difference to enable the microorganism or micro-particle to be transferred to the transferring module.

13. The apparatus of claim 6, wherein the first sterilization module corresponds to at least one of an ultraviolet lamp and a sterilization filter.

14. The apparatus of claim 8, wherein the second sterilization module corresponds to at least one of an ultraviolet lamp and a sterilization filter.

15. A method of detecting a microorganism or micro-particle in real time, the method comprising:
electrically charging a microorganism or micro-particle in an atmosphere;
collecting the electrically charged microorganism or micro-particle using a potential difference;
preprocessing the collected microorganism or micro-particle by applying a fluorescent material to the collected microorganism or micro-particle;
irradiating a light;
sensing a microorganism or micro-particle which reacts to the light; and
detecting a concentration of the microorganism or micro-particle in the atmosphere.

16. The method of claim 15, further comprising:
filtering a microorganism or micro-particle which is larger than a predetermined size from the atmosphere.

17. The method of claim 15, further comprising:
transferring the collected microorganism or micro-particle to a transfer belt.

18. The method of claim 15, further comprising:
sterilizing the microorganism or micro-particle, which is adhered to a predetermined collection module, in a first sterilizing operation; and
separating the microorganism or micro-particle, which is not transferred from the collection module to a predetermined transferring module, from the collection module in a first cleaning operation.

19. The method of claim 17, further comprising:
sterilizing the transferred microorganism or micro-particle in a second sterilizing operation; and
separating the microorganism or micro-particle from a predetermined transferring module in a second cleaning operation.

20. A computer-readable recording medium storing a program for implementing a method of detecting a microorganism or micro-particle in real time, the method comprising:
electrically charging a microorganism or micro-particle in an atmosphere;
collecting the electrically charged microorganism or micro-particle using a potential difference;
preprocessing the collected microorganism or micro-particle by applying a fluorescent material to the collected microorganism or micro-particle;
irradiating a light;
sensing a microorganism or micro-particle which reacts to the light; and
detecting a concentration of the microorganism or micro-particle in the atmosphere.
